(19) United States
(12) Patent Application Publication
(10) Pub. No.: US 2009/0326078 A1
(43) Pub. Date: Dec. 31, 2009

(54) PROCESS FOR PREPARING A SOLID DOSAGE FORM

(75) Inventors: Jean Paul Remon, Melle (BE); Chris Vervaet, Izegem (BE); Yves Gonnissen, Tongeren-Sluizen (BE)

Correspondence Address: CLARK & ELBING LLP, 101 FEDERAL STREET, BOSTON, MA, 02110, US

(73) Assignee: Universiteit Gent, Gent (BE)

(21) Appl. No.: 12/306,717

(22) PCT Filed: Jun. 27, 2007
(86) PCT No.: PCT/EP07/05673

§ 371 Date: Dec. 26, 2008

(30) Foreign Application Priority Data
Jun. 27, 2006 (GB) 0612695.7

(51) Int. Cl. A61K 47/26; A61K 47/10

(52) U.S. Cl. 514/778; 514/772

(57) ABSTRACT

This invention provides a process for preparing a solid dosage form, comprising:—preparing an aqueous slurry, solution or suspension of (a) a powder material, and (b) a mixture of one or more polyols and one or more maltodextrins, and—spray drying the resultant aqueous slurry, solution or suspension, thereby obtaining particles which are directly compressible into a solid dosage form being able to disintegrate in an aqueous medium within no more than 15 minutes.

This invention relates to the field of particle design technology. In particular, it relates to a process for the preparation, preferably the continuous preparation, of a solid dosage form. In the pharmaceutical area, it is more particularly useful for making compressed tablets, in particular rapidly disintegrating tablets with a high drug loading. This invention also relates to compressible compositions for making a solid dosage form which is able to disintegrate in an aqueous medium within a short time, e.g. a limited number of minutes at room temperature.

### BACKGROUND OF THE INVENTION

Powders intended for compression into tablets for the pharmaceutical and healthcare industries must possess two essential properties: fluidity and compressibility. Fluidity is required so that the material can be transported through the hopper of a tableting machine and so that adequate filling of the dies occurs in the tableting machine to produce tablets of a consistent weight. Although powder flow can be improved mechanically by the use of vibrators, the latter can cause powder segregation and stratification. Powder flow properties can also be increased by incorporating minute amounts of a glidant such as fumed silicium dioxide or by granulation. Compressibility is the property of forming a stable, intact compact mass when pressure is applied. Some materials are known to compact better than others, e.g. paracetamol is poorly compressible whereas lactose compresses well, however as a general rule granulation improves compressibility. The same concerns apply to detergent powders intended for making high-density detergent granules and for compression into detergent pressings.

Limited use of spray-drying has been made for producing particles which are directly compressible into a solid dosage form. Sustained release over a time period of several hours has been achieved with a solid dosage form obtained by directly compressing particles obtained by spray-drying a slurry of an active agent admixed with microcrystalline cellulose in intimate association with silicon dioxide or a surfactant acting as a compressibility increasing agent in amounts up to 50% by weight of microcrystalline cellulose. Drug tablets which disintegrate within less than one minute have been obtained by tableting a mixture of a drug, a lubricant (e.g. stearic acid or a salt thereof) and particles obtained by spray-drying a slurry of a disintegrating agent (preferably crospovidone, hydroxypropylcellulose, croscarmellose sodium or crystalline cellulose), an inorganic excipient (preferably a silicate, phosphate, carbonate or hydroxide containing aluminium, magnesium or calcium) and certain carbohydrates.

However, in certain situations where spray-drying is preferred over granulation for technical and/or economical reasons as a method for preparing directly compressible particles, there is a need in the art for designing compressible compositions for making a solid dosage form which is able to disintegrate in an aqueous medium within a short time, e.g. a limited number of minutes at room temperature. More specifically in the pharmaceutical industry, there is a need in the art for a process for preparing, preferably continuously preparing, a solid dosage form such as rapidly disintegrating compressed tablets whatever the drug loading in said tablets, especially when the tablet involves a high drug loading.

### SUMMARY OF THE INVENTION

The present invention is based on the first unexpected finding that a solid dosage form, such as a tablet, with a controlled disintegration time in an aqueous medium can easily be made by directly compressing particles obtained by spray drying an aqueous slurry or solution of (a) a powder material and (b) a mixture of one or more polyols and a maltodextrin. More specifically, the present invention makes it possible to overcome the various above mentioned problems while providing a solid dosage form which is able to disintegrate in an aqueous medium within a short time, e.g. a limited number of minutes at room temperature. The present invention is widely applicable to any kind of powder material such as, but not limited to, a pharmaceutical or veterinary ingredient, an agrochemical ingredient, a fertiliser or a plasticiser.

### DETAILED DESCRIPTION OF THE INVENTION

The present invention will be described with reference to certain embodiments and drawings but the present invention is not limited thereto but only by the attached claims. The following working embodiments are given by way of example only.

A first object of the present invention is a process for preparing a solid dosage form, comprising:
preparing an aqueous slurry, solution or suspension of (a) a powder material, and (b) a mixture of one or more polyols and one or more maltodextrins, and
spray drying the resultant aqueous slurry, solution or suspension, thereby obtaining particles which are directly compressible into a solid dosage form being able to disintegrate in an aqueous medium within a controlled but short period of time, preferably within no more than about 15 minutes at room temperature.

The exact type and the operating conditions of the spray drying equipment or device to be used in the spray drying step of the process of the invention are not limiting features of the present invention. There is no particular limitation for the condition of spray-drying. In the (preferred) spray drying step the aqueous slurry, solution or suspension is brought together with a sufficient volume of hot air to produce evaporation and drying of the liquid droplets. The highly dispersed slurry, solution or suspension is pumpable and capable of being atomized. With regard to a spray-dryer, it is preferred to use a spray-dryer of a disk type or a nozzle type. With regard to the temperature for spray-drying, it is preferred that the inlet temperature into the spray drying equipment is from about 100 to about 400° C., e.g. from about 100 to about 300° C. and/or that the outlet temperature outwards from the spray drying equipment is from about 50 to about 200° C., e.g. from about 50 to about 130° C. It is preferred that the aqueous slurry be dried using spray-drying techniques, as they are known in the art. Other drying techniques, however, such as flash drying, ring drying, micron drying, tray drying, vacuum drying, radio-frequency drying, and possibly microwave drying, may also be used alternatively, although spray drying is preferred. Depending upon parameters such as, but not limited to, the type of drying conditions used, the concentration of components (a) and (b) in the aqueous slurry, solution or suspension, the novel directly compressible particles of this invention may have different useful properties such as particle size, density, moisture content, etc.

In one particular embodiment of the process of the invention, the aqueous slurry, solution or suspension may further comprise a disintegrating agent. The exact nature and the amount of disintegrating agent to be present in the aqueous slurry, solution or suspension submitted to the process of the invention are not limiting features of the present invention. Suitable disintegrating agents include, but are not restricted to, crospovidone, low-substituted hydroxypropyl cellulose and croscarmellose sodium and, although any of them may be used solely, it is also permissible to use a mixture of two or more thereof. The disintegrating agent preferably includes crospovidone as a main such agent. Suitable amounts of a disintegrating agent are usually in the range of about 0.5% by weight to about 20% by weight, preferably from about 2% by weight to about 12% by weight, based on solids content in the aqueous slurry, solution or suspension.

In one particular embodiment of the process of the invention, the aqueous slurry, solution or suspension may further comprise a lubricant. The exact nature and the amount of the lubricant to be present in the aqueous slurry, solution or suspension submitted to the process of the invention are not limiting features of the present invention. Suitable lubricants include, but are not restricted to, stearic acid and salts thereof, such as magnesium stearate, calcium stearate, and stearyl fumarate sodium. Suitable amounts of a lubricant are usually in the range of about 0.2% by weight to about 2% by weight, preferably from about 0.4% by weight to about 1% by weight, based on solids content in the aqueous slurry, solution or suspension.

In one particular embodiment of the process of the invention, the aqueous slurry, solution or suspension may further comprise one or more surfactants or tensio-active agents. The exact nature and the amount of the one or more surfactants to be optionally present in the aqueous slurry, solution or suspension prepared and submitted to spray drying according to the process of the present invention are not limiting features of the present invention. Suitable surfactants for this purpose are described below. Suitable amounts of such tensio-active agents are usually in the range of about 0.1% by weight to about 5% by weight, preferably from about 0.5% by weight to about 3% by weight, based on solids content in the aqueous slurry, solution or suspension, depending upon the type of surfactant being used.

In one particular embodiment of the present invention, the aqueous slurry, solution or suspension may further comprises one or more binders. The exact nature and the amount of the one or more binders optionally present in the aqueous slurry, solution or suspension prepared and submitted to spray drying according to this embodiment of the process are not limiting features of the present invention. Suitable binders include, but are not limited to, polymers and cellulose derivatives. Representative but non-limiting examples thereof include hydroxypropyl cellulose, hydroxyethyl cellulose, carboxymethylcellulose, ethylcellulose, methylcellulose, hydroxypropylmethyl cellulose phthalate, microcrystalline cellulose, starch, lactose, acacia, dextrin, gelatin, glucose, guar gum, polymethacrylates, sodium alginate, and mixtures thereof. The one or more binders may be present in this embodiment in an effectively binding amount of up to about 10% by weight, in particular an amount ranging from about 0.1 to 4% by weight. More particularly, this embodiment is advantageous when the powder material has a low compressibility, and/or when the powder material is highly dosed, e.g. constitutes more than about 70% by weight, or more than about 80% by weight, or more than about 90% by weight, or more than about 95% by weight, of the solid dosage form being prepared. A low compressibility, as defined herein, refers to a tablet friability not above 1% by weight when the solid dosage form is a tablet.

In one particular embodiment of the process of the invention, the aqueous slurry, solution or suspension may further comprises one or more process yield increasing agents. The exact nature and the amount of the process yield increasing agent to be optionally present in the aqueous slurry, solution or suspension prepared and submitted to spray drying according to the process of the invention are not limiting features of the present invention. Suitable process yield increasing agents include, but are not restricted to, glidants such as colloidal silicon dioxide. Suitable amounts of a process yield increasing agent are usually in the range of about 0.1% by weight to about 5% by weight, preferably from about 0.2% by weight to about 3% by weight, based on solids content in the aqueous slurry, solution or suspension.

The exact nature and the amount of the one or more polyols to be present in the aqueous slurry, solution or suspension prepared and submitted to spray drying according to the process of the invention are not limiting features of the present invention. However, depending upon parameters such as, but not limited to, the type of polyol, the concentration of the one or more polyols in the aqueous slurry, solution or suspension, and their proportion with respect to the maltodextrin, the novel directly compressible particles of this invention may have different useful properties such as particle size, density, moisture content, etc. Depending upon the same parameters, the solid dosage forms, such as (but not limited to) tablets or hard capsules, resulting from the direct compression of such particles may have different useful properties such as friability, hardness, etc. In view of optimisation of such properties, it is often preferred when said one or more polyols include, or are selected from the group consisting of, mannitol, erythritol, and mixtures thereof in any proportions.

The exact nature and the amount of the maltodextrin(s) to be present in the aqueous slurry, solution or suspension prepared and submitted to spray drying according to the process of the invention are not limiting features of the present invention. However, depending upon parameters such as, but not limited to, the type of maltodextrin, its concentration in the aqueous slurry, solution or suspension, and its proportion with respect to the one or more polyols, the novel directly compressible particles of this invention may have different useful properties such as particle size, density, moisture content, etc. As is well known to the skilled person, the different types of maltodextrins are mainly defined with respect to the two following parameters:
the amylose and amylopectin contents of maltodextrin; in practice the amylose content commonly ranges from about 0.1 to about 70% by weight of maltodextrin and the amylopectin content commonly ranges from about 30 to about 99.9% by weight of maltodextrin; and
the dextrose equivalent (herein referred as DE) of maltodextrin, which commonly ranges from about 1 to about 20.

The respective proportions of the one or more polyols with respect to the one or more maltodextrins may vary within wide ranges, depending upon other parameters such as the weight ratio of the powder material (a) to the mixture (b). According to one embodiment of the present invention, the weight ratio of the one or more polyols to the one or more maltodextrins in the mixture (b) preferably ranges from about 1:1 to about 5:1, more preferably from about 1.5:1 to about 4:1.

Depending upon the same parameters, the solid dosage forms such as (but not limited to) tablets and hard capsules resulting from the direct compression of such compressible particles may have different useful properties such as friability, hardness, etc. In view of optimisation of such properties, it is also permissible to use a mixture of one or more maltodextrins of different types such as referred herein-above.

Suitable but non-limiting examples of the weight ratio of the powder material (a) to the mixture (b) are from about 1:10000 to about 100:1, preferably from about 1:100 to about 10:1, more preferably from about 1:10 to about 5:1.

The exact solids content in the aqueous slurry, solution or suspension prepared and submitted to spray drying according to the process of the invention is not a limiting feature of the present invention. The solids content of said aqueous slurry, solution or suspension prior to said spray drying step may be for instance from about 1% to about 50% by weight, e.g. from about 2% to about 35% by weight.

The exact nature and the amount of the powder material (a) to be present in the aqueous slurry, solution or suspension prepared and submitted to spray drying according to the process of the invention are not limiting features of the present invention. Depending upon the intended use for the solid dosage form resulting from the process, said powder material (a) may be, among others, a pharmaceutical or veterinary ingredient, an agrochemical ingredient, a fertiliser or a plasticizer. A number of illustrative examples thereof are given below. The amount of the powder material (a) may be such that it constitutes from about 30% by weight to about 95% by weight of the solid dosage form. In particular, the present invention is advantageous when said powder material (a) is a substance, e.g. a pharmaceutical or veterinary ingredient, with low compressibility and/or when said powder material (a) is highly dosed, e.g. is present in an amount of at least about 70% by weight of the solid dosage form.

The exact nature and the amount of the liquid phase of the aqueous slurry, solution or suspension prepared and submitted to spray drying according to the process of the invention are not limiting features of the present invention. The liquid is preferably a substance or mixture of substances which does not alter, or chemically interfere with, the substantial properties of the powder material to be processed. Therefore the liquid phase is usually selected according to the characteristics, such as moisture sensitivity, of the specific powder material concerned. For economic and safety reasons, water is usually preferred as the main component of the liquid phase, but lower alcohols such as methanol, ethanol or isopropanol, or mixtures thereof with water in various proportions, may constitute suitable alternatives when moisture sensitivity is a limitation inherent to said powder material. When the powder material is a pharmaceutical or veterinary ingredient, the components of the liquid phase are preferably selected from pharmaceutically acceptable grade components.

In a further embodiment, the process of the invention may further comprise a step of compressing the directly compressible particles obtained from spray drying into a solid dosage form such as, but not limited to, a tablet or a hard capsule. This possibility is due to the excellent flowability and cohesiveness of such compressible particles.

Another aspect of the present invention relates to a particulate compressible composition suitable for making a solid dosage form, such as (but not limited to) a tablet or a hard capsule, being able to disintegrate in an aqueous medium within a controlled but short period of time, preferably within no more than about 15 minutes at room temperature, said composition comprising (a) a powder material, (b) a mixture of one or more polyols and one or more maltodextrins.

In one embodiment the particulate compressible composition according to the invention may further comprise one or more disintegrating agents. The exact nature and the amount of the one or more disintegrating agents to be optionally present in the particulate compressible composition according to the invention are not limiting features of this other aspect of the present invention, as described herein-before with respect to the process of manufacture of said directly compressible particles.

In one embodiment the particulate compressible composition according to the invention may further comprise one or more lubricants. The exact nature and the amount of the one or more lubricants to be optionally present in the particulate compressible composition of the invention are not limiting features of this other aspect of the present invention, as described hereinbefore with respect to the process of manufacture of said directly compressible particles.

In another embodiment the particulate compressible composition according to the invention may further comprise one or more surfactants. The exact nature and the amount of the one or more surfactants to be present in the particulate compressible composition of the invention are not limiting features of this other aspect of the present invention, as described hereinbefore with respect to the process of manufacture of said directly compressible particles.

In another embodiment, the particulate compressible composition according to the invention may further comprise one or more process yield increasing agents. The exact nature and the amount of the one or more process yield increasing agents to be optionally present in the particulate compressible composition of the invention are not limiting features of this other aspect of the present invention, as described herein-before with respect to the process of manufacture of said directly compressible particles.

The exact nature and the amount of the one or more polyols to be present as part of the mixture (b) in the particulate compressible composition of the invention are not limiting features of this other aspect of the present invention, as described herein-before with respect to the process of manufacture of said directly compressible particles. Polyols selected from the group consisting of mannitol and erythritol are however preferred in many circumstances.

The exact nature and the amount of the maltodextrin to be present as part of the mixture (b) in the particulate compressible composition of the invention are not limiting features of this other aspect of the present invention, as described herein-before with respect to the process of manufacture of said directly compressible particles.

The exact nature and the amount of the powder material to be present as a component (a) in the particulate compressible composition of the invention are not limiting features of the present invention, as described herein-before with respect to the process of manufacture of said directly compressible particles. Suitable but non limiting examples of the powder material (a) include a pharmaceutical or veterinary ingredient, an agrochemical ingredient, a fertiliser and a plasticiser. A suitable weight ratio of the powder material (a) to the mixture (b) of one or more polyols and a maltodextrin is from about 1:10000 to about 100:1, preferably from about 1:100 to about 10:1, more preferably from about 1:10 to about 5:1.

In yet another aspect the present invention relates to a solid dosage form, such as (but not limited to) a tablet or a hard capsule, being able to disintegrate in an aqueous medium within a controlled but short period of time, preferably within no more than about 15 minutes, said solid dosage form being made being compressing a particulate compressible composition defined or prepared such as described herein-above with respect to the other aspects of the invention. The aqueous medium in which quick disintegration of the solid dosage form of this invention occurs is a predominantly based on water, but may also include minor amounts of other water-miscible or water-dispersible components, depending upon the exact nature of the powder material and the intended use and field of action of the solid dosage form. For instance, when the powder material (a) is a pharmaceutically or veterinary ingredient for administration to a mammal body, including a human being, the aqueous medium may be any biological fluid present in the said mammal body. The temperature at which disintegration of the solid dosage form of this invention occurs may also be relevant to the determination of the disintegration speed.

The relevant temperature is also dependent upon the exact nature of the powder material (a) and the intended use and field of action of the solid dosage form, and can be easily determined by the skilled person. For instance, when the powder material (a) is a pharmaceutically or veterinary ingredient for administration to a mammal body, including a human being, the relevant temperature is close to room temperature or body temperature, i.e. within a range from about 20° C. to about 37° C. When the powder material (a) is a detergent, the relevant temperature is close to the temperature at which said detergent is admixed with the usually aqueous dispersing or washing medium.

Due to its constitution and its method of manufacture, the solid dosage form according to the invention, in particular a tablet, is able to exhibit an improved friability, e.g. a tablet friability not above about 1% by weight.

The chemical constitution, particle size, or any other physical property, of the powder material (a) to be used in the process and composition of this invention are not critical parameters. The powder material may be selected for instance from foodstuffs, mineral ores, agricultural products (e.g. fertilisers), detergents, catalysts, chemicals, as well as biologically active ingredients and compositions containing the latter together with one or more suitable conventional additives, modifiers or excipients as may be relevant to the intended use or field of industry.

Examples of foodstuffs suitable as a powder material (a) include, but are not limited to, animal foodstuff such as vitamins, proteins, lipids, sugars, cellulose for poultry, fish, pigs, dogs, cats, and cattle in general, as well as human foodstuff such as flower, sugar, instant preparations for soup or puddings.

Examples of catalysts suitable as a powder material (a) include, but are not limited to, any type of zeolites or catalysts, including for instance catalysts suitable for packing fluidised bed reactors.

Examples of detergents suitable as a powder material (a) include, but are not limited to, those containing typical ingredients for detergents, for example water-soluble emulsifiers and synthetic surface-active agents including anionic and non-ionic surfactants (such as also defined below with regard to pharmaceutically acceptable excipients), builders, inorganic electrolytes, re-deposition inhibitors, foam inhibitors, bleaches and bleach activators, optical brighteners, enzymes, fabric softeners and dyes and fragrances. Suitable organic and inorganic builders for detergents are soluble and/or insoluble components which show a mildly acidic, neutral or alkaline reaction and which are capable of precipitating or complexing calcium ions. Suitable and, in particular, ecologically safe builders are e.g. finely crystalline, synthetic water-containing zeolites of the NaA type in detergent quality. Their particle size is normally in the range from 1 to 10 μm. Their content is generally from 0 to 40% by weight, of the detergent composition, based on anhydrous substance. Other builders which may be used in particular together with said zeolites include (co)polymeric polycarboxylates, such as polyacrylates, polymethacrylates and, in particular, copolymers of acrylic acid with about 50% to 10% maleic acid and an average molecular weight from about 50,000 to 100,000. Suitable, but less preferred compounds of this class are copolymers of at least about 50% acrylic or methacrylic acid with vinyl ethers, such as vinyl methyl ether. Other organic builders are e.g. non-polymeric polycarboxylic acids preferably used in the form of their sodium salts, such as citric acid or nitrilo-triacetic acid. Suitable inorganic electrolytes are the bicarbonates, carbonates, borates or silicates of the alkali metals also known as “washing alkalis ”. Suitable re-deposition inhibitors for detergent compositions, being able to keep the soil separated from the fibers suspended in the wash liquor, are water-soluble, generally organic colloids such as e.g. the water-soluble salts of polymeric carboxylic acids, glue, gelatine, salts of ethercarboxylic acids or ether-sulfonic acids or acidic sulfuric acid esters of cellulose or starch. Water-soluble polyamides containing acidic groups, soluble starch preparations (e.g. degraded starch or aldehyde starches), polyvinylpyrrolidone, carboxymethyl cellulose (sodium salt), methyl cellulose, methyl hydroxyethylcellulose and mixtures thereof are also suitable. Foam inhibitors include, but are not limited to, soaps, preferably natural and synthetic soaps having a high content of C₁₈-C₂₄ fatty acids, organopolysiloxanes, paraffins, waxes, microcrystalline waxes and mixtures thereof with silanized silica. Suitable bleaches include, but are not limited to, sodium perborate tetra- or monohydrate, peroxycarbonates, peroxypyrophosphates, citrate perhydrates and peracidic salts or peracids, such as perbenzoates, peroxophthalates, diperazelaic acid or diperdodecanedioic acid. Suitable bleach activators include N-acyl and O-acyl compounds such as N,N′-tetraacylated diamines, and carboxylic anhydrides and esters of polyols such as glucose pentaacetate. Suitable optical brighteners include derivatives or alkali metal salts of diaminostilbene disulfonic acid such as 4,4′-bis-(2-anilino-4-morpholino-1,3,5-trazin-6-ylamino)-stilbene-2,2,-disulfonic acid or similar compounds which, instead of the morpholino group, contain a diethanolamino group, a methylamino group, an anilino group or a 2-methoxyethylamino group. Suitable enzymes may be selected from proteases, lipases, amylases and mixtures thereof, e.g. as obtained from bacterial strains or fungi such as Bacillus subtilis, Bacillus licheniformis and Streptomyces griseus, and may be adsorbed onto carriers and/or encapsulated into shell-forming substances in order to protect them against premature decomposition.

The term “biologically active ingredient” as used herein with respect to a suitable powder material (a) refers to therapeutic, diagnostic, cosmetic or prophylactic pharmaceutical and veterinary agents as well as other agents, e.g. selected from insecticides, pesticides, herbicides, plant growth regulators, fertilisers, crop treatment agents, anti-microbial agents (in particular fungicides and bactericides), admissible for use in plants, animals and humans. Thus the biologically active compositions made according to this invention may be for pharmaceutical use, cosmetic use, veterinary use or for plant treatment. The therapeutic agent can be selected for its specific properties such as for instance its anti-thrombotic, anti-inflammatory, anti-proliferative or anti-microbial efficiency. The latter include for instance anti-microbial agents such as broad spectrum antibiotics for combating clinical and sub-clinical infection, for example gentamycin, vancomycine and the like. Other suitable therapeutic agents are naturally occurring or synthetic organic or inorganic compounds well known in the art, including non-steroidal anti-inflammatory drugs, proteins and peptides (that may be produced either by isolation from natural sources or through recombination), hormones (for example androgenic, estrogenic and progestational hormones such as oestradiol), bone repair promoters, carbohydrates, antineoplastic agents, antiangiogenic agents, vasoactive agents, anticoagulants, immunomodulators, cytotoxic agents, antiviral agents, antibodies, neurotransmitters, oligonucleotides, lipids, plasmids, DNA and the like.

Suitable therapeutically active proteins include e.g. fibroblast growth factors, epidermal growth factors, platelet-derived growth factors, macrophage-derived growth factors such as granulocyte macrophage colony stimulating factors, ciliary neurotrophic factors, tissue plasminogen activator, B cell stimulating factors, cartilage induction factor, differentiating factors, growth hormone releasing factors, human growth hormone, hepatocyte growth factors, immunoglobulins, insulin-like growth factors, interleukins, cytokines, interferons, tumor necrosis factors, nerve growth factors, endothelial growth factors, osteogenic factor extract, T cell growth factors, tumor growth inhibitors, enzymes and the like, as well as fragments thereof.

Suitable diagnostic agents include conventional imaging agents (for instance as used in tomography, fluoroscopy, magnetic resonance imaging and the like) such as transition metal chelates. Suitable anti-microbial agents include e.g. halogenated phenols, chlorinated diphenylethers, aldehydes, alcohols such as phenoxyethanol, carboxylic acids and their derivatives, organometallic compounds such as tributyltin compounds, iodine compounds, mono- and polyamines, sulfonium and phosphonium compounds; mercapto compounds as well as their alkaline, alkaline-earth and heavy metal salts; ureas such as trihalocarbanilide, isothia- and benzisothiazolone derivatives.

Suitable insecticides include natural ones, e.g. nicotine, rotenone, pyrethrum and the like, and synthetic ones like chlorinated hydrocarbons, organophosphorus compounds, biological insecticides (e.g. products derived from Bacillus thuringiensis), synthetic pyrethroids, organosilicon compounds, nitro-imines and nitromethylenes. Suitable fungicides include e.g. dithiocarbamates, nitrophenol derivatives, heterocyclic compounds (including thiophtalimides, imidazoles, triazines, thiadiazoles, triazoles and the like), acylalanines, phenylbenzamides and tin compounds.

Suitable herbicides include e.g. trichloroacetic and aromatic carboxylic acids and their salts, substituted ureas and triazines, diphenyl ether derivatives, anilides, uraciles, nitriles and the like.

Suitable fertilizers include e.g. ammonium sulphate, ammonium nitrate, ammonium phosphate and the like, and mixtures thereof.

Therapeutically active agents which may be advantageously incorporated into the directly compressible particles of the present invention preferably belong to all permeability and solubility classes of the Biopharmaceutical Classification System according to G. Amidon et al. in Pharm. Res. (1995) 12:413-420, in particular the two classes of poorly soluble drugs, i.e. Class II and Class IV of the said classification. As will be appreciated by those skilled in the art, these drugs belong to various therapeutic classes including, but are not limited to, beta-blockers, calcium antagonists, ACE inhibitors, sympathomimetic agents, hypoglycaemic agents, contraceptives, α-blockers, diuretics, anti-hypertensive agents, anti-psoriatics, bronchodilators, cortisones, anti-mycotic agents, salicylates, cytostatic agents, antibiotic agents, virustatic agents, antihistamines, UV-absorbers, chemotherapeutics, antiseptics, estrogens, scar treatment agents, anti-fungal agents, antibacterial agents, antifolate agents, cardiovascular agents, nutritional agents, antispasmodics, analgesics, antipyretics, anti-inflammatory agents, coronary vasodilators, peripheral vasodilators, anti-tussive agents, muscle relaxants, tranquilisers, antiarrythmic agents, anticoagulants, anti-emetics, expectorants, anti-diabetic agents and the like.

This invention is suitable e.g. for (but not limited to) preparing solid dosage forms of one or more of the following therapeutically active ingredients or cosmetic agents: acebutolol, acetohexamide, acetylcysteine, acetylsalicylic acid, acyclovir, ajamaline, alendronate, alfuzosine, alprazolam, alfacalcidol, allantoin, allopurinol, alverine, ambroxol, amikacin, amlodipine, amiloride, aminoacetic acid, amiodarone, amitriptyline, amlodipine, amoxicillin, ampicillin, amylobarbitone, ascorbic acid, aspartame, astemizole, atenolol, beclomethasone, benserazide, benzalkonium hydrochloride, benzocaine, benzoic acid, betamethasone, bezafibrate, biotin, biperiden, bisoprolol, bromazepam, bromhexine, bromocriptine, budesonide, bufexamac, buflomedil, buspirone, caffeine, camphor, captopril, carbamazepine, carbidopa, carboplatin, cefachlor, cefalexin, cefatroxil, cefazolin, cefixime, cefotaxime, ceftazidime, ceftriaxone, cefuroxime, cephalosporins, cetirizine, chloramphenicol, chlordiazepoxide, chlorhexidine, chlorpheniramine, chlortalidone, choline, cyclosporin, cilastatin, cimetidine, ciprofloxacin, cisapride, cisplatin, citalopram, clarithromycin, clavulanic acid, clomipramine, clonazepam, clonidine, clotrimazole, codeine, cholestyramine, cromoglycic acid, cyanocobalamin, cyproterone, desogestrel, dexamethasone, dexpanthenol, dextromethorphan, dextropropoxiphen, diazepam, diclofenac, digoxin, dihydrocodeine, dihydroergotamine, dihydroergotoxin, diltiazem, diphenhydramine, dipyridamole, dipyrone, disopyramide, domperidone, dopamine, doxycycline, enalapril, ephedrine, epinephrine, ergocalciferol, ergotamine, erythromycin, estradiol, ethinylestradiol, etoposide, Eucalyptus globulus, famotidine, felodipine, fenofibrate, fenoterol, fentanyl, flavine mononucleotide, fluconazole, flunarizine, fluorouracil, fluoxetine, flurbiprofen, furosemide, gallopamil, gemfibrozil, Ginkgo biloba, glibenclamide, glipizide, clozapine, Glycyrrhiza glabra, griseofulvin, guaifenesin, haloperidol, heparin, hyaluronic acid, hydrochlorothiazide, hydrocodone, hydrocortisone, hydromorphone, ipratropium hydroxide, ibuprofen, imipenem, indomethacin, iohexol, iopamidol, isosorbide dinitrate, isosorbide mononitrate, isotretinoin, ketotifen, ketoconazole, ketoprofen, ketorolac, labetalol, lactulose, lecithin, levocarnitine, levodopa, levoglutamide, levonorgestrel, levothyroxine, lidocaine, lipase, imipramine, lisinopril, loperamide, lorazepam, lovastatin, medroxyprogesterone, menthol, methotrexate, methyldopa, methylprednisolone, metoclopramide, metoprolol, miconazole, midazolam, minocycline, minoxidil, misoprostol, morphine, N-methylephedrine, naftidrofuryl, naproxen, neomycin, nicardipine, nicergoline, nicotinamide, nicotine, nicotinic acid, nifedipine, nimodipine, nitrazepam, nitrendipine, nizatidine, norethisterone, norfloxacin, norgestrel, nortriptyline, nystatin, ofloxacin, omeprazole, ondansetron, pancreatin, panthenol, pantothenic acid, paracetamol, paroxetine, penicillins, phenobarbital, pentoxifylline, phenoxymethylpenicillin, phenylephrine, phenylpropanolamine, phenytoin, physostigmine, piroxicam, polymyxin B, povidone iodine, pravastatin, prazepam, prazosin, prednisolone, prednisone, bromocriptine, propafenone, propranolol, proxyphylline, pseudoephedrine, pyridoxine, quinidine, ramipril, ranitidine, reserpine, retinol, riboflavin, rifampicin, risperidone, rutoside, saccharin, salbutamol, salcatonin, salicylic acid, simvastatin, somatotropin, sotalol, spironolactone, sucralfate, sulbactam, sulfamethoxazole, sulfasalazine, sulpiride, tamoxifen, tegafur, teprenone, terazosin, terbutaline, terfenadine, tetracaine, tetracycline, theophylline, thiamine, ticlopidine, timolol, tranexamic acid, tretinoin, triamcinolone acetonide, triamterene, triazolam, trimethoprim, troxerutin, uracil, valproic acid, verapamil, folinic acid, zidovudine, zopiclone, enantiomers thereof, organic and inorganic addition salts (including acid salts and base salts) thereof, solvates (such as hydrates and alcoholates) thereof and mixtures thereof, in particular mixtures in synergistic proportions.

Other biologically active ingredients useful for performing this invention are vitamins, including those of the A group, of the B group (which means, besides B1, B2, B6 and B12, also compounds with vitamin B properties such as adenine, choline, pantothenic acid, biotin, adenylic acid, folic acid, orotic acid, pangamic acid, carnitine, p-aminobenzoic acid, myo-inositol and lipoic acid), vitamin C, vitamins of the D group, E group, F group, H group, I and J groups, K group and P group.

The present invention is also suitable for the formulation of therapeutically active ingredients (drugs) having a water-solubility below about 2.5 mg/ml, even between 0.1 and 1 mg/ml (i.e. “very slightly soluble” as defined in the United States Pharmacopeia), even below 0.1 mg/ml (i.e. “practically insoluble” as defined in the United States Pharmacopeia), even below about 5 μg/ml and may even have a water-solubility as low as about 0.2 μg/ml, at room temperature and physiological pH. Non-limiting examples of such drugs include for instance hydrochlorothiazide, nimodipine, flufenamic acid, mefenamic acid, bendroflumethiazide, benzthiazide, ethacrinic acid, nitrendipine and diaminopyrimidines, including enantiomers thereof, organic and inorganic addition salts (including acid salts and base salts) thereof, and solvates (such as hydrates and alcoholates) thereof. Suitable examples of such poorly soluble diaminopyrimidines include, without limitation, 2,4-diamino-5-(3,4,5-trimethoxybenzyl) pyrimidine (trimethoprim), 2,4-diamino-5-(3,4-dimethoxy-benzyl) pyrimidine (diaveridine), 2,4 diamino-5-(3,4,6-trimethoxybenzyl) pyrimidine, 2,4-diamino-5-(2-methyl-4,5-dimethoxybenzyl) pyrimidine (ormeto-prim), 2,4-diamino-5-(3,4-dimethoxy-5-bromobenzyl) pyrimidine, 2,4-diamino-5-(4-chloro-phenyl)-6-ethylpyrimidine(pyrimethamine), and analogues thereof.

This invention is suitable for the direct compression of formulations including said biologically active ingredients (e.g. drugs) together with one or more physiologically (e.g. pharmaceutically) acceptable excipients such as, but not limited to, emulsifiers or surface-active agents, thickening agents, gelling agents or other additives, and wherein the active ingredient (e.g. drug) loading, i.e. the proportion or content of the active ingredient (e.g. drug) in the formulation, may vary through wide ranges. For instance said active ingredient content may be at least about 0.1% by weight, preferably at least 1% by weight, for example at least 5% by weight. Furthermore, said active ingredient content in the final formulation (solid dosage form) may also be up to about 70% by weight, for instance at most 40% by weight, for example at most 30% by weight.

Emulsifiers, surfactants or surface-active agents suitable for therapeutically active formulations or other powder material compositions according to this invention include, but are not limited to, water-soluble natural soaps and water-soluble synthetic surface-active agents or surfactants. Suitable soaps include alkaline or alkaline-earth metal salts, non-substituted or substituted ammonium salts of higher, preferably saturated, fatty acids (C₁₀-C₂₂), e.g. the sodium or potassium salts of oleic or stearic acid, or of natural fatty acid mixtures obtainable form coconut oil, palm oil or tallow oil. Synthetic surface-active agents (surfactants) include anionic, cationic and non-ionic surfactants, e.g. sodium or calcium salts of polyacrylic acid; sulfonated benzimidazole derivatives preferably containing 8 to 22 carbon atoms; alkylarylsulfonates; and fatty sulfonates or sulphates, usually in the form of alkaline or alkaline-earth metal salts, non-substituted ammonium salts or ammonium salts substituted with an alkyl or acyl radical having from 8 to 22 carbon atoms, e.g. the sodium or calcium salt of lignosulfonic acid or dodecylsulfonic acid or a mixture of fatty alcohol sulphates obtained from natural fatty acids, alkaline or alkaline-earth metal salts of sulphuric or sulfonic acid esters (such as sodium lauryl sulphate) and sulfonic acids of fatty alcohol/ethylene oxide adducts. Examples of alkylarylsulfonates are the sodium, calcium or alcanolamine salts of dodecylbenzene sulfonic acid or dibutylnaphthalene-sulfonic acid or a naphthalene-sulfonic acid/formaldehyde condensation product. Also suitable are the corresponding phosphates, e.g. salts of phosphoric acid ester and an adduct of p-nonylphenol with ethylene and/or propylene oxide) and the like.

Suitable emulsifiers further include, but are not limited to, partial esters of fatty acids (e.g. lauric, palmitic, stearic or oleic) or hexitol anhydrides (e.g., hexitans and hexides) derived from sorbitol, such as commercially available polysorbates. Other emulsifiers which may be used include, but are not limited to, adducts of polyoxyethylene chains (1 to 40 moles ethylene oxide) with non-esterified hydroxyl groups of the above partial esters, such as the surfactant commercially available under the trade name Tween 60 from ICI Americas Inc.; and the poly(oxyethylene)/poly(oxypropylene) materials marketed by BASF under the trade name Pluronic.

Suitable structure-forming, thickening or gel-forming agents for the biologically active compositions of this invention include, but are not limited to, highly dispersed silicic acid, such as the product commercially available under the trade name Aerosil; bentonites; tetra-alkyl ammonium salts of montmorillonites (e.g. products commercially available under the trade name Bentone) wherein each of the alkyl groups may contain from 1 to 20 carbon atoms; ceto-stearyl alcohol and modified castor oil products (e.g. a product commercially available under the trade name Antisettle).

Gelling agents which may be included into the biologically active ingredient compositions of the present invention include, but are not limited to, cellulose derivatives such as carboxymethylcellulose, cellulose acetate and the like; natural gums such as arabic gum, xanthum gum, tragacanth gum, guar gum and the like; gelatin; silicium dioxide; synthetic polymers such as carbomers, and mixtures thereof. Gelatin and modified celluloses represent a preferred class of gelling agents.

Hydrophilic cellulose derivatives may also be used as pharmaceutically acceptable excipients for formulating the therapeutically active particulate compositions according to the invention, in particular as a binder optionally present in the particulate compressible composition. The term “hydrophilic ” herein refers to a cellulose derivative or polymer having groups, preferably non-ionizable groups, that are capable of hydrogen bonding, in particular of association with water molecules at physiologically relevant pH. Suitable examples of hydrophilic cellulose polymers that can be used in the present invention include, but are not limited to, polymers having ether-linked substituents, for instance hydroxy-alkylalkylcelluloses (wherein the alkyl group preferably has from 1 to 4 carbon atoms) such as hydroxypropylmethylcellulose, i.e. cellulose 2-hydroxypropyl methyl ether (hereinafter referred to as HPMC). It is a non-ionic water-soluble ether of methylcellulose which is insoluble in hot water but dissolves slowly in cold water. Being used extensively as a drug tablet excipient, HPMC is commercially available under various trade names. Suitable grades of HPMC include a low viscosity grade such as Methocel K100 from Dow Chemical, a high viscosity grade such as Methocel K100M, and other types such as the Metolose 90SH series from Shinetsu.

Amphiphilic materials may be used as well as pharmaceutically acceptable excipients for formulating therapeutically active particulate compositions according to the invention. The term “amphiphilic” herein refers to a material having both a hydrophobic portion, for instance comprising aliphatic or aromatic hydrocarbon groups, and a hydrophilic portion. Suitable examples of such amphiphilic materials include, but are not limited to, those having both a portion derived from a glyceride and a portion derived from a polyethylene glycol ester. For instance, it is suitable to use polyglycosylated glycerides as an amphiphilic material excipient in the present invention. The expression “polyglycosylated glycerides” as used herein denotes a mixture of mono-, di- and triglycerides with polyethylene glycol (PEG) mono- and diesters of C₈-C₁₈ fatty acids with a molecular weight preferably between about 200 and about 600, optionally further including glycerol and/or free PEG, the hydrophilic-lipophilic balance (HLB) value of which is controlled by the chain length of the PEG and the melting point of which is controlled by the chain length of the fatty acids, of the PEG and of the degrees of saturation of the fatty chains, and thus of the starting oil. Similarly the expression “C₈-C₁₈ fatty acids” as used herein denotes mixtures in various proportions of caprylic acid, capric acid, lauric acid, myristic acid, palmitic acid and stearic acid, when these acids are saturated, and the corresponding unsaturated acids. As is well known to the skilled person, the proportions of these fatty acids may vary as a function of the starting oils. Examples of the latter include, but are not limited to, saturated polyglycolized C₈-C₁₀ glycerides, such as the PEG-8 caprylate-caprate glyceride esters sold by Gattefosse Corporation under the tradename Labrasol; PEG-6 caprylic/capric glycerides sold by Huls Aktiengesellschaft under the trade name Softigen 767; PEG-60 corn glycerides sold by Croda under the trade name Crovol M-70; Ceteareth-20 sold by Henkel Corporation under the trade name Eumulgin B2; diethyleneglycol monoethyl-ethers sold by Gattefosse Corporation under the trade name Transcutol; a mixture of C₈-C₁₈ saturated polyglycosylated glycerides having a melting point within a range of about 42-48° C. and a HLB within a range of about 8 to 16 such as sold by Gattefosse Corporation under the trade names Gelucire 48/09, Gelucire 44/14 and Gelucire 42/12; and mixtures thereof in various proportions.

Other optional excipients which may also be present in the biologically active compositions made according to the present invention include (but are not limited to) one or more additives such as magnesium oxide; azo dyes; organic and inorganic pigments such as titanium dioxide; UV-absorbers; stabilisers; odor masking agents; viscosity enhancers; antioxidants such as, for example, ascorbyl palmitate, sodium bisulfite, sodium metabisulfite and the like, and mixtures thereof; preservatives such as, for example, potassium sorbate, sodium benzoate, sorbic acid, propyl gallate, benzyl alcohol, methyl paraben, propyl paraben and the like; sequestering agents such as ethylene-diamine tetra-acetic acid; flavoring agents such as natural vanillin; buffers such as citric acid or acetic acid; extenders or bulking agents such as silicates, diatomaceous earth, magnesium oxide or aluminum oxide; densification agents such as magnesium salts; sweeteners; and mixtures thereof.

When the biologically active particulate formulation of the invention is intended for making an effervescent solid dosage form, it should necessarily include sodium bicarbonate and one or more weak acids, such as citric acid or tartaric acid, acting as a carbon dioxide liberator. Such effervescent formulations can be made for the purpose of effervescent tablets, e.g. for cleaning artificial teeth.

The selection of the optimal excipients and their proportion in the biologically active particulate formulations of the present invention depends, in a manner which is well known to the skilled person, on a series of parameters such as, but not limited to, the specific biologically-active ingredient to be formulated, the end-user requirements (in particular cost), the load (i.e. weight proportion) of the biologically-active ingredient in the solid dosage form, and the required biologically-active ingredient (e.g. drug) release characteristics (in particular kinetics).

The process for making solid dosage forms according to the present invention, as well as the particulate compressible compositions resulting therefrom provide inter alia the following advantages:
technical and economical improvement in the long-term operating conditions, in particular for drug formulations, pharmaceutical compositions and foodstuffs, by avoiding lubrication problems, reducing power consumption, avoiding excessive temperatures which could be detrimental to the powder material to be formulated, and increasing the total processing yield, while eliminating the need for intermediate steps before compaction,
use of a standard equipment suitable for various powder materials, including those which are difficult to manufacture with other available techniques, such as detergents, drug formulations of all kinds and foodstuffs,
a technology which is fully compatible with the Good Manufacturing Principles of the pharmaceutical industry,
manufacturing cost effectiveness due to the use of standard equipment and the lack of unnecessary intermediate steps, and
making particles from pharmaceutical compositions and excipients which, when directly compacted into tablets, provide improved tablet properties, in particular improved tensile strength and hardness, and improved friability, together with a quick disintegration profile in an aqueous medium.

In view of the above technical and economical advantages, the various aspects of the present invention are useful and extremely valuable to the pharmaceutical industry, the chemical industry, the detergent and mining industries, and the foodstuff industry.

The following examples are provided for illustrative purpose only, and should in no way be interpreted as limiting the scope of the present invention.

### EXAMPLES 1 to 12

### Making and Tableting Compressible Drug-Containing Particles by Spray-Drying

Aqueous slurries were prepared including a drug substance selected from (A) acetaminophen, (B) Ibuprofen and (C) cimetidine; mannitol; erythritol; maltodextrin; a disintegrant (crospovidone); optionally a glidant (colloidal silicon dioxide); optionally a lubricant (magnesium stearate); optionally a binder (hydroxypropyl methylcellulose, also referred to as HPMC) and a surfactant (polysorbate 80) in weight proportions as shown in the following table. The corresponding tablet compositions are shown in the following table.

Powder mixtures were formulated by co-processing spray drying these aqueous slurries. Spray drying was performed in a pilot plant scale equipment, available under the trade name Mobile Minor, Model D Special from NIRO, Copenhagen, Denmark. The slurries were fed to an atomisation device (two-fluid nozzle, pressure nozzle, rotary atomiser) of the spray dryer by means of a peristaltic pump. The spray dryer was operating in co-current or counter-current air flow. Finally the spray dried particles were collected in a reservoir attached to a cyclone, cooled down to room temperature and sieved over a 375-μm sieve and stored prior to their characterisation and compaction into tablets.

Compaction of the compressible drug particles obtained from spray drying into 500 mg tablets was effected on a single punch tablet press. The directly compressible powder mixtures were compacted on an excentric tablet press (type EKO, commercially available from Korsch, Berlin, Germany) equipped with 13.5 mm edged punches. The tablet properties of all solid dosage forms were evaluated at a different compression force of 9.8 kN (74 MPa) for examples 1-6, 18.6 kN (130 MPa) for examples 7-9, 12.3 kN (86 MPa) for examples 10-11, and 17.2 kN (120 MPa) for example 12. For each compact, 500 mg of powder was weighed on an analytical balance, and then manually filled into the die.

The resulting tablets have been investigated for critical properties including:
hardness or diametral crushing force, expressed in Newtons, using a tester type PTB available from Pharma Test, Hainburg, Germany;
friability, expressed as a percentage by weight, tested using a friabilator, (type PTF available from Pharma Test, Hainburg, Germany); the value indicated in the following Table is an average calculated from tests performed on 10 tablets;
disintegration time, expressed in minutes and seconds, determined as follows. A total of 6 tablets were tested simultaneously for disintegration time using a disintegrator, type PTZ available from Pharma Test, Hainburg, Germany. The test was performed using 900 ml demineralised water maintained at 37° C. as immersion fluid.

The corresponding results for these properties are provided in the following table.

TABLE

| Example | 1 | 2 | 3 | 4 | 5 | 6 |
|---|---|---|---|---|---|---|
| Drug substance | A | A | A | A | A | A |
| solid content in slurry (% w/w) | 3.1 | 2.8 | 3.1 | 9.3 | 18.6 | 28.0 |
| Spray dried composition (% w/w) | | | | | | |
| Drug Substance | 41.4 | 46.0 | 42.1 | 41.9 | 41.9 | 41.9 |
| Mannitol | 8.3 | 9.2 | 8.8 | 20.9 | 20.9 | 20.9 |
| Erythritol | 20.7 | 23.0 | 21.1 | 14.2 | 14.2 | 14.2 |
| Maltodextrin (Glucidex ® 9) | 12.4 | | 6.3 | 10.0 | 10.0 | 10.0 |
| Maltodextrin (Glucidex ® 2) | | 14.2 | | | | |
| Maltodextrin (Novelose ® 330) | | | 6.3 | | | |
| Crospovidone (Kollidon ® CL) | 12.0 | 4.0 | 12.0 | 12.0 | 12.0 | 12.0 |
| Colloidal silicon dioxide | 1.5 | 0.5 | 0.5 | 0.5 | 0.5 | 0.5 |
| Magnesium stearate | 1.0 | 0.5 | 0.7 | | | |
| Polysorbate 80 | 2.7 | 2.7 | 2.5 | 0.5 | 0.5 | 0.5 |
| Tablet properties | | | | | | |
| (diameter: 13.5 mm) | | | | | | |
| Hardness (N) | 51.2 | 93.1 | 37.7 | 106.0 | 83.1 | 73.2 |
| Friability (% w/w) | 0.73 | 0.78 | 0.8 | 0.85 | 0.98 | 1.00 |
| Disintegration time | 7′30″ | 11′50″ | 5′10″ | 7′04″ | 2′52″ | 2′26″ |
| (minutes & seconds) | | | | | | |

| Example | 7 | 8 | 9 | 10 | 11 | 12 |
|---|---|---|---|---|---|---|
| Drug substance | A | A | A | B | B | C |
| solid content in slurry (% w/w) | 37.8 | 40.0 | 35.0 | 35.0 | 30.0 | 37.8 |
| Spray dried composition (% w/w) | | | | | | |
| Drug Substance | 48.7 | 60.0 | 85.0 | 60.0 | 75.0 | 65.0 |
| Mannitol | 20.6 | 15.3 | 3.0 | 15.3 | 8.4 | 12.1 |
| Erythritol | 14.0 | 10.4 | 2.1 | 10.4 | 5.7 | 8.2 |
| Maltodextrin (Glucidex ® 9) | 9.8 | 7.3 | 1.4 | 7.3 | 4.0 | 5.8 |
| HPMC (Metolose ® SR 60SH-50) | | | 2.0 | | | |
| Crospovidone (Kollidon ® CL) | 6.0 | 6.0 | 6.0 | 6.0 | 6.0 | 5.9 |
| Colloidal silicon dioxide | 0.5 | 0.5 | | 0.5 | 0.5 | 0.5 |
| Magnesium stearate | | | | | | 2.0 |
| Polysorbate 80 | 0.5 | 0.5 | 0.5 | 0.5 | 0.5 | 0.5 |
| Tablet properties | | | | | | |
| (diameter: 13.5 mm) | | | | | | |
| Hardness (N) | 76.6 | 65.8 | 65.6 | 94.6 | 89.0 | 93.4 |
| Friability (% w/w) | 0.76 | 0.87 | 0.59 | 0.46 | 0.47 | 0.81 |
| Disintegration time | 5′36″ | 3′37″ | 6′43″ | 11′00″ | 9′20″ | 4′40″ |
| (minutes & seconds) | | | | | | |

1-28. (canceled)

29. A process for preparing a solid dosage form, comprising:
preparing an aqueous slurry, solution or suspension of (a) a powder material, wherein said powder material is an active pharmaceutical or veterinary ingredient and (b) a mixture of one or more polyols selected from the group consisting of mannitol, erythritol and mixtures thereof and one or more maltodextrins, and
spray drying the resultant aqueous slurry, solution or suspension, thereby obtaining particles which are directly compressible into a solid dosage form being able to disintegrate in an aqueous medium within no more than 15 minutes.

30. A process according to claim 29, wherein the solids content of said aqueous slurry, solution or suspension prior to said spray drying step is from 1% to 50% by weight.

31. A process according to claim 29, wherein the weight ratio of the powder material (a) to the mixture (b) is from 1:10000 to 100:1.

32. A particulate compressible composition suitable for making a solid dosage form being able to disintegrate in an aqueous medium within no more than 15 minutes, comprising (a) a powder material, wherein said powder material is an active pharmaceutical or veterinary ingredient and (b) a mixture of one or more polyols selected from the group consisting of mannitol and erythritol and one or more maltodextrins.

33. A particulate compressible composition according to claim 32, further comprising one or more disintegrating agents.

34. A particulate compressible composition according to claim 32, further comprising one or more lubricants.

35. A particulate compressible composition according to claim 33, further comprising one or more lubricants.

36. A particulate compressible composition according to claim 32, further comprising one or more surfactants.

37. A particulate compressible composition according to claim 33, further comprising one or more surfactants.

38. A particulate compressible composition according to claim 34, further comprising one or more surfactants.

39. A particulate compressible composition according to claim 32, further comprising one or more process yield increasing agents.

40. A particulate compressible composition according to claim 32, further comprising one or more binders.

41. A particulate compressible composition according to claim 33, further comprising one or more binders.

42. A particulate compressible composition according to claim 40, wherein said one or more binders is present in an amount of up to 4% by weight.

43. A particulate compressible composition according to claim 41, wherein said one or more binders is present in an amount of up to 4% by weight.

44. A particulate compressible composition according to claim 40, wherein said powder material has low compressibility and/or constitutes more than 70% by weight of said solid dosage form.

45. A particulate compressible composition according to claim 32, wherein the weight ratio of the powder material (a) to the mixture (b) is from 1:10000 to 100:1.

46. A particulate compressible composition according to claim 32, wherein the weight ratio of the one or more polyols to the one or more maltodextrins in the mixture (b) ranges from 1:1 to 5:1.

47. A solid dosage form being able to disintegrate in an aqueous medium within no more than 15 minutes, being made being compressing a particulate compressible composition according to claim 32.

48. A solid dosage form according to claim 47, being a tablet with a friability not above 1% by weight.
